(19) United States
(12) Patent Application Publication
(10) Pub. No.: US 2003/0036720 A1
(43) Pub. Date: Feb. 20, 2003

(54) Hemodialysis assembly and method

(76) Inventors: Dudley W.C. Spencer, Wilmington, DE (US); Ivars V. Ivansons, Wilmington, DE (US)

Correspondence Address: Connolly Bove Lodge & Hutz LLP, P.O. Box 2207, Wilmington, DE, 19899-2207, US

(21) Appl. No.: 10/222,663

(22) Filed: Aug. 16, 2002

Related U.S. Application Data
(60) Provisional application No. 60/313,042, filed on Aug. 17, 2001.

(51) Int. Cl. A61M 37/00

(52) U.S. Cl. 604/6.04

(57) ABSTRACT

A multi-stage cell segregation from the plasma by staged separation and rinsing processes is performed while the plasma is diverted to the dialyzer and de-watering steps are completed without the cells being present. Cleaning the plasma of the urea and other life threatening toxins and pathogens permits a faster and more thorough cleaning of the plasma and cells which in turn reduces the therapy time.

### CROSS REFERENCE TO RELATED APPLICATION

This application is based upon provisional application Ser. No. 60/313,042 filed Aug. 17, 2001.

### BACKGROUND OF THE INVENTION

A typical patient undergoing hemo-dialysis receives 3 treatments a week, each lasting 4 to 6 hours depending upon various patient related conditions. During each treatment the patient's total blood volume is drawn from the patient, passed through a urea separation device and returned in a continuous fashion to the patient.

Many clinical complications arise because of the need to remove urea and other toxins while the fragile cells are present: of these, the compatibility of the dialyzer membranes and whole blood is of major concern. Both proteins and cells adhere to the membrane surface. This contact, in turn, causes a number of deleterious responses, which resemble those of assaults to the patient's immune system in the form of severe allergic reactions, serious compliment activation in the C₃ C₅ region, which the body reacts as if invaded by a foreign pathogen. The responses are numerous and in some instances life threatening. They include:

Platelet adhesion, agglomeration & damage,

Thrombosis and erythrocyte adhesion.

Complement activation (C₃/C₅)

The clinical manifestations are also numerous and in some cases lead to morbidity and mortalities.

Anemia

Hypoxemia

Organ damage

Blood poisoning (Septicemia)

Infections, lumen & surface penetration

Malignancies

Leukopenia

### SUMMARY OF THE INVENTION

It is the objective of the invention to reduce or eliminate these life-threatening consequences of the basic life saving process of dialysis, while at the same time reduce the time required to cleanse the patient's blood.

### THE DRAWING

FIGS. 1 is a schematic view of the continuous spindialysis principle;

FIG. 2 is a schematic view of a multistage spin dialysis practice of this invention; and

FIG. 3 is a schematic view showing one of the stages of plasma removal and red blood cell mixing.

### DETAILED DESCRIPTION

The present application is based upon variations in application Ser. Nos. 09/329,269 filed Jun. 28, 1999, and 09/510,634 filed Feb. 22, 2000, now both abandoned. All of the details of those applications are incorporated herein by reference thereto. All of the details of co-pending application Ser. No. 10/177,038, filed Feb. 15, 2002, are also incorporated herein by reference thereto.

A preferred practice of the invention utilizes a multi-tiered cell separation, cell washing technique followed by a plasma cleaning process (without cells present) and a re-combination of cells and plasma: which leads to a significant reduction in patient assaults brought in by existing processes and a possible 25% reduction of Patient-on-machine therapy time.

For physiological reasons, the time period required for currently practiced dialysis is somewhat fixed. Modeling the removal process as a classical first-order kinetics

C═Co*e^{-k/v*t}.

Where k/v is the fractional removal rate per time period. For a “normal patient” with 40 liters of body water and 43% Hct, assuming a blood flow limit of 350 ml/min, the optimum shortest treatment time; therefore, for existing processes would be about four hours.

The new simplified concept can be performed continuously in the following manner:

Whole blood is removed from the patient at a rate of 350 ml/mm.

The cells are separated from the plasma and rinsed of all contaminates.

The urea is removed from the plasma.

The rinsed cells and cleaned plasma are recombined.

Blood is returned to the patient continuously.

To maintain sterility, avoiding the threat of patient infection, a sterile connection device, such as Denco's Sterile Connection Device TCD® Model B-40 can be used.

The net effect of cell and plasma cleaning, using processes conducive to the specific needs of cells and plasma independently results not only in cleaner blood being returned to the patient but in approx 25-30% less time.

Over time, as the process matures, addition advances will be found to further reduce therapy time.

The concept of the invention utilizes three basic operations:

1. Separation of the cells from the plasma and cell rinsing to gently wash the cells.

2. Removal of the urea and other toxins & Pathogens from the plasma.

3. Re-combining the cells and the plasma.

The first operation, cell separation and rinsing, is easily done using a continuous process.

The second operation, urea, toxin and Pathogen removal, can be performed in the same manner as existing hemodialysis, diffusion through a membrane. As this is also a continuous process, it can be easily matched to a continuous cell separation process.

The third operation, recombination, is a new process. While not difficult, it is likely that the invention can accomplish this on a continuous basis.

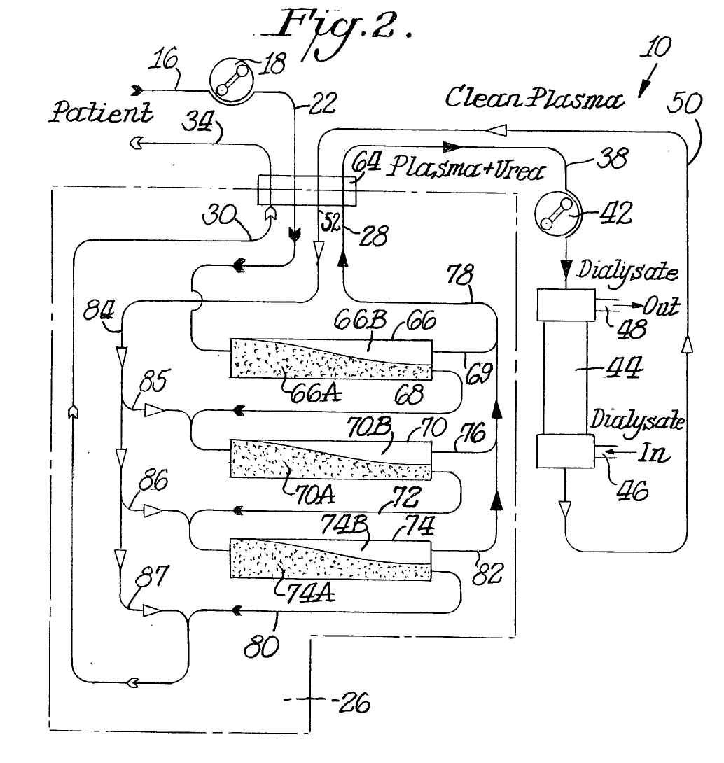

As illustrated in FIG. 2, a multi-stage cell segregation from the plasma by staged separation and rinsing processes, while the plasma is diverted to the dialyzer and de-watering steps are completed without the cells being present. Cleaning the plasma of the urea and other life-threatening toxins and pathogens permits a faster and more thorough cleaning of the plasma and cells, which in turn results in the reduction of therapy time of 4 hrs to 3 or less.

As is known, after many years of membrane research the dialysis process as now practiced, is rate dependant and has reached a point where cell destruction and membrane diffusion has reached its limit.

Spin-dialysis, as used herein, is a new and novel departure from the known art and represents a breakthrough in dialysis technology to better serve the needs of those whose lives depend upon dialysis.

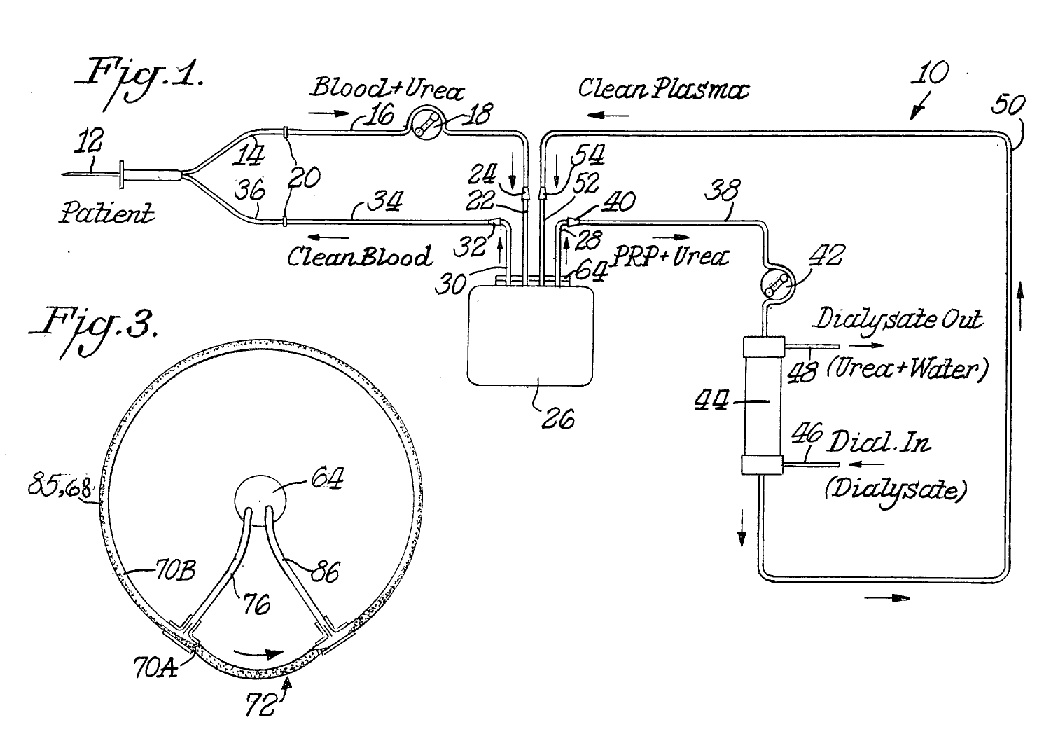

FIG. 1 schematically illustrates the continuous spin dialysis principle in accordance with this invention. As shown therein, the continuous spin dialysis system 10 would include, for example, a patient catheter 12 which would be inserted into the patient for facilitating the removal of anticoagulated blood and urea through line or tube 14. Tube 14 is connected to a further tube or passageway 16 which is associated with blood pump 18 for removing the blood and urea. The invention could be practiced with a single continuous tube extending from the catheter. Two separate tubes are preferred, however, to permit the catheter 12 to remain in the patient without most of the remaining portions of the assembly hooked to the patient. Tubes 14 and 16 could be effectively connected or disconnected by a sterile connection device to form a joint 20 or to permit separation at the joint 20. Thus, the catheter 12 can remain in the patient with discharge tube 14 and return tube 36 detached from the remainder of system 10 at times when the system is not in use.

As used herein the term “sterile connection device” is intended to refer to a device having a pair of aligned spaced tube holders each of which has at least one tube receiving channel and includes a heated wafer located to pass between the spaced tube holders for heating plastic tubes therein. The connection or separation of such plastic tube is done in a sterile manner. Such sterile connection devices may be of the type described in U.S. Pat. Nos. 4,793,880, 5,141,592, 5,256,229, 5,279,685 and 5,674,333, all of the details of which are incorporated herein by reference thereto.

Tube 16 is in flow communication with tube 22 and the tubes 16,22 may be coupled together at location 24 in any suitable manner including by means of a sterile connection device. Tube 22 leads into a continuous cell separation centrifuge 26 which is better shown in FIGS. 2-4. The tubing, such as tubes 14, 16 and 22, heading to centrifuge 26 may be considered as feed tubing. Thus, the anticoagulated blood and urea is conveyed from the patient to the centrifuge 26 where the platelet rich plasma (PRP) and urea or other substances are removed from centrifuge 26 into tube 28. The remaining clean blood having red cells and white cells is conveyed from centrifuge 26 into return tube 30. Tube 30 is connected at location 32 to return tube 34 which in turn communicates with return tube 36 so that the cleaned blood is returned to the patient. As indicated, tubes 34 and 36 may have a sterile connection 20.

The plasma and urea are conveyed from tube 28 into tube 38. Plasma conveying tubes 28 and 38 may be connected together at any suitable location 40 in any suitable manner. Pump 42 operates to withdraw the PRP and urea from centrifuge 26 and discharge the PRP and urea into dialyzer 44. As indicated a dialysate is introduced to dialyzer 44 through tube or line 46. The dialysate and contaminates such as urea and water are discharged from dialyzer 44 through tube or line 48. The clean platelet rich plasma is conveyed from dialyzer 44 through discharge tube 50 back into centrifuge 26 by means of connecting tube 52 which is connected to tube 50 at location 54 in any suitable manner.

FIG. 2 schematically illustrates the process in practicing this invention with system 10. As shown in FIG. 3 the anticoagulated blood and urea would be fed through tube 16 and tube 22 through rotary seal 64 into centrifuge 26. Blood pump 18 could operate at 300 ml/min. The anticoagulated blood and urea would then enter the centrifuge first ring stage 66 flowing, for example, at 300 ml/min. In the first ring stage 66 the red blood cells packed with HCT collects at region 66A while the plasma and urea and other contaminates collect in region 66B. The red blood cells are removed from ring stage 66 and conveyed through tube 68 into second ring stage 70. For example, the flow could be 114 ml/min RBC's packed at 80% HCT (38% of flow). The plasma and urea could flow from ring stage 66 through tube 69 at 186 m/min which would be 62% of the flow. Outlet tube 69 communicates with tube 78 which is in flow communication with tube 28.

The packed red blood cells would flow from tube 68 and mix with clean plasma coming in from tube 85. Because urea is permeable to the blood cell membranes, the clean plasma will dilute and rinse the urea from the red blood cells prior to entering into second ring stage 70. The red blood cells, under centrifuge force, would then collect in region 70A while the plasma and urea would collect in region 70B. The packed blood cells would flow from the second ring stage 70 through tube 72, mix with clean plasma from tube 86 and enter the third ring stage 74. Similarly, the plasma and urea would flow from region 70B of the second ring stage 70 through outlet tube 76 and meet with the plasma and urea flowing from the first stage 66 through tube 69 into a common tube 78. The diluted blood from the second stage entering the third ring stage 74 would again separate into plasma and urea in region 74B and packed blood cells in region 74A. The packed cells would be discharged from the third stage into tube 80 and mixed with clean plasma from tube 87, from a final rinse, and then returned back to the patient through tube 30. The plasma and urea from the third ring stage separation 74 would be discharged through outlet tube 82 which would join with tube 76 and 69 which in turn joins with outlet tube 78 ultimately being directed to the dialyzer 44.

In the first cycle, for example, 62% of the urea would be removed. In the second cycle 87% of the urea would be removed. In a later third cycle 96% of the urea would be removed.

The clean plasma would return from the dialyzer 44 through tube 50 into the centrifuge through rotary seal 64 and tube 52. Tube 52 communicates with tube 84 which branches off to join tubes 68, 72 and 80 so that the cleaned plasma can mix with the separated and packed blood cells and subject it to further cleaning by dilution. The clean plasma, which joins with the packed blood cells through tube 80, restores the blood to its original hematocrit HCT 30% level prior to leaving the centrifuge 26 through tube 30.

FIG. 3 illustrates the detail of one of the stages, such as stage 70 shown in FIG. 2. As illustrated in FIG. 3 the packed RBC's and urea would enter second stage 70. The red blood cell sump 70A would be located below the plasma space 70B of FIG. 2. Clean plasma mixed with red blood cells would flow from tube 84 to tube 68 as shown in FIG. 2 so that the combination of clean plasma from dialyzer 44 and the red blood cells from region 66A of first stage 66 would be fed into second stage 70. The plasma and urea would be discharged from second stage 70 through tube 76 (as shown in FIG. 3) and ultimately to tube 28 which passes through the center manifold and rotary seal 64 as shown in FIGS. 2 and 3. Similarly, the dialyzed plasma from dialyzer 44 would flow through tube 50 through center manifold and rotary seal 64 into tube 52, 84, and 86 as also shown in FIGS. 2 and 3.

What is claimed is:

1. A system for separating blood cells from plasma comprising feed tubing communicating with a centrifuge for conveying blood from a patient to said centrifuge, said centrifuge being a multi-stage centrifuge for separating plasma and contaminates from the cells in the blood, plasma tubing extending from said centrifuge to a dialyzer for conveying the blood and contaminates from said centrifuge to said dialyzer, said dialyzer comprising structure for separating the plasma from the contaminates without the cells being present, return tubing leading from said centrifuge for conveying the blood cells from said centrifuge to the patient, and discharge tubing communicating with said dialyzer for conveying the separated plasma away from said dialyzer back to said centrifuge.

2. The system of claim 1 said discharge tubing communicates with branch tubing within said centrifuge for conveying the separated plasma into at least one of the stages in said centrifuge along with the blood cells being conveyed to at least one of said stages.

3. The system of claim 2 wherein said branch tubing conveys the separated plasma to a plurality of the stages in said centrifuge.

4. The system of claim 3 wherein said branch tubing includes a branch communicating directly with said return tubing for conveying separated plasma into said return tubing to mix with the blood cells.

5. The system of claim 4 wherein said multi-stage centrifuge includes at least three stages for successively separating the blood cells from the plasma and contaminates.

6. The system of claim 5 wherein each of said stages is connected to an adjacent stage by cell conveying tubing, and each of said stages including outlet tubing communicating with said plasma tubing for conveying plasma from each of said stages to said dialyzer.

7. A system for separating blood cells from plasma comprising feed tubing communicating with a centrifuge for conveying blood from a patient to said centrifuge, said centrifuge being a multi-stage centrifuge for separating plasma and contaminates from the cells in the blood, plasma tubing extending from said centrifuge to a dialyzer for conveying the blood and contaminates from said centrifuge to said dialyzer, said dialyzer comprising structure for separating the plasma from the contaminates without the cells being present, return tubing leading from said centrifuge for conveying the blood cells from said centrifuge to the patient, discharge tubing communicating with said dialyzer for conveying the separated plasma away from said dialyzer and back to said centrifuge, said discharge tubing communicating with branch tubing within said centrifuge for conveying the separated plasma into a plurality of the stages in said centrifuge along with the blood cells being conveyed to said plurality of said stages.

8. The system of claim 7 wherein said branch tubing includes a branch communicating directly with said return tubing for conveying separated plasma into said return tubing to mix with the blood cells.

9. The system of claim 8 wherein said multi-stage centrifuge includes at least three stages for successively separating the blood cells from the plasma and contaminates.

10. The system of claim 9 wherein each of said stages is connected to an adjacent stage by cell conveying tubing, and each of said stages including outlet tubing communicating with said plasma tubing for conveying plasma from each of said stages to said dialyzer.

11. A method for separating blood cells from plasma comprising conveying blood from a patient to a multi-stage centrifuge and into the first stage of the centrifuge, subjecting the blood to a first separation to partially separate blood cells from plasma and other contaminates, conveying the partially separated blood cells successively to at least one additional stage for further separation of the blood cells from the plasma and contaminates, returning the separated blood cells to the patient, conveying the separated plasma and contaminates from each stage to a dialyzer, separating the plasma from the contaminates in the dialyzer without the cells being present, conveying the separated plasma from the dialyzer back to the centrifuge, and feeding at least some of the separated plasma to at least one of the stages.

12. The method of claim 11 including feeding the separated plasma to a multiple of the stages.

13. The method of claim 12 including feeding some of the separated plasma directly to tubing which returns the separated blood cells to the patient to mix the separated plasma and separated blood cells so that a mixture thereof is returned to the patient.
